(19) United States
(12) Patent Application Publication
(10) Pub. No.: US 2007/0265307 A1
(43) Pub. Date: Nov. 15, 2007

(54) Administration of 4-aminopiperidine compounds for inducing and/or stimulating the growth of keratin fibers and/or preventing loss thereof

(75) Inventors: Alexandre Cavezza, Pavillon Sous Bois (FR); Philippe Breton, Noisy Le Roi (FR); Maria Dalko, Gif S/yvette (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/790,450

(22) Filed: Apr. 25, 2007

Related U.S. Application Data
(60) Provisional application No. 60/799,332, filed on May 11, 2006.

(30) Foreign Application Priority Data
Apr. 25, 2006 (FR) 0651451

(51) Int. Cl. A61K 31/445; A61K 8/49

(52) U.S. Cl. 514/317

(57) ABSTRACT

Agents for inducing and/or stimulating the growth of human keratin fibers and/or preventing their loss and/or increasing their density include at least one 4-aminopiperidine compound of formula (I):

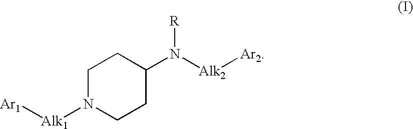

### CROSS-REFERENCE TO PRIORITY/PROVISIONAL APPLICATIONS

This application claims priority under 35 U.S.C. § 119 of FR 06/51451, filed Apr. 25, 2006, and of U.S. Provisional Application No. 60/799,332, filed May 11, 2006, each hereby expressly incorporated by reference and each assigned to the assignee hereof.

### CROSS-REFERENCE TO COMPANION APPLICATION

Copending U.S. Patent Application No. ______ [Attorney Docket No. 1016800-000801], filed concurrently herewith and also assigned to the assignee hereof.

### BACKGROUND OF THE INVENTION

1. Technical Field of the Invention

The present invention relates to the formulation of 4-aminopiperidine compounds of specific formula (I) into care or makeup compositions for human keratin fibers, especially for topical application, useful to induce and/or stimulate the growth of keratin fibers and/or prevent their loss. This invention also relates to a cosmetic treatment to stimulate the growth of keratin fibers and/or prevent their loss.

More especially, the present invention relates to the formulation of an effective amount of a 4-aminopiperidine compound of specific formula (I) into care or makeup compositions for the hair or the eyelashes, useful to increase their density and/or improve their appearance.

2. Description of Background and/or Related and/or Prior Art

Hair growth and hair renewal are mainly determined by the activity of the hair follicles and by their matrix environment. Their activity is cyclic and essentially comprises three phases, namely the anagenic phase, the catagenic phase and the telogenic phase.

The anagenic phase (active phase or growth phase), which lasts several years and during which the hair gets longer, is followed by a very short and transient catagenic phase which lasts a few weeks. During this phase, the hair undergoes a change, the follicle becomes atrophied and its implantation in the dermis appears higher and higher.

The terminal phase or telogenic phase, which lasts a few months, corresponds to a rest phase for the follicle and the hair falls out. At the end of this rest period, a new follicle is regenerated, in place, and another cycle begins again.

The head of hair is thus in constant renewal and, out of the approximately 150,000 hairs which make up a head of hair, approximately 10% of them are at rest and will be replaced within a few months.

The natural loss of the hair can be estimated, on average, at a few hundred hairs per day for a normal physiological state. This process of constant physical renewal undergoes a natural change during aging, and the hairs become finer and their cycles shorter.

In adulthood, the vascular system of the skin is complete and no longer changes, except in the hair follicles, where it undergoes considerable changes with each hair cycle. Specifically, the hair follicles are a richly innervated and highly vascularized cutaneous structure. The phenomenon of development of capillary circulation in the hair follicles is known as angiogenesis. At the beginning of each anagenic phase, it is necessary to develop high activation of angiogenesis in order to redevelop the perifollicular vascular capillary network. The involution of this capillary network and the disappearance of the blood vessels of the dermal papilla go hand in hand with the change of phase and the passage into the catagenic phase. At this stage, the blood capillaries collapse and disappear.

In parallel, in the alopecic areas, a perifollicular fibrosis becomes established, the follicles reduce in size cycle after cycle and the specific vascularization of the bulbs gradually diminishes.

The phenomenon of angiogenesis observed during the anagenic phase is dependent on many trophic factors, cytokines or other biologically active molecules provided by the blood stream or produced locally, in particular by the fibroblasts of the dermal papilla or the keratinocytes of the hair bulb. Among these trophic factors, mention may be made of endothelial cell growth factor (also known as vascular endothelial growth factor (VEGF)). This factor is essential for angiogenesis and increases vascular permeability. Studies have shown that the expression of this factor is increased during the anagenic phase of the hair cycle. Thus, this factor contributes towards maintaining functional capillary vascularization around the hair follicle, and in particular at the base of the bulb and of the dermal papilla, and also towards supplying nutrients required for good growth of the hair.

The perifollicular capillary circulation thus plays a fundamental role in the process of hair growth by supplying the factors and nutrients required for the growth of this follicle.

Hair loss may be greatly accentuated and the follicle renewal cycles may be highly disrupted in certain dermatoses of the scalp with an inflammatory component, for example psoriasis or seborrheic dermatitis.

Other causes may result in substantial, temporary or permanent hair loss. This may involve hair loss or impairment at the terminal stage of a pregnancy (post-partum), during states of denutrition or malnutrition, physiological stress or dietary imbalances or else during states of asthenia or of hormonal dysfunction, as may be the case during or at the terminal stage of the menopause. It may also involve hair loss or impairments related to seasonal phenomena.

It may also be a matter of alopecia, which is essentially due to a disturbance in hair renewal, resulting, in a first stage, in acceleration of the frequency of the cycles to the detriment of the quality of the hair, and then of the quantity thereof. This then results in a gradual impoverishment of the head of hair and in gradual thinning of the hair together with isolation of the bulbs due to progressive thickening of the perifollicular collagen matrix and of the outer connective sheath. Revascularization is thus made more difficult cycle after cycle. The successive growth cycles result in hairs that are finer and finer and shorter and shorter, gradually transforming into an unpigmented down. Some areas are preferentially affected, in particular the temporal or frontal lobes in men, and a diffuse alopecia of the crown of the head is observed in women.

By virtue of the essential role of the perifollicular capillary circulation stated above, any deficiency in the latter will result in a decrease in the supply of the nutritive and gaseous (oxygen, in particular) elements required for hair growth, resulting in disturbances in growth of the hair and the gradual establishment of alopecia.

The term “alopecia” also covers a whole family of afflictions of the hair follicle whose final consequence is the permanent, partial or general loss of the hair. This is more particularly termed androgenic alopecia. In a large number of cases, early hair loss occurs in genetically predisposed individuals; this is then termed androchronogenetic alopecia. This form of alopecia especially affects men.

In certain dermatoses of the scalp with an inflammatory component, for example psoriasis or seborrheic dermatitis, hair loss can be greatly accentuated or can result in highly disturbed follicular cycles.

In general, any factor that results in an increase in blood supply in the hair follicle, either by activating angiogenesis or by opposing regression thereof, or else by acting on the capillary vessels to limit their constriction, will have a beneficial effect on the energy supply required for good growth of this same follicle.

The cosmetic or pharmaceutical industry has for a number of years been seeking compositions that make it possible to eliminate or reduce alopecia, and in particular to induce or stimulate hair growth and decrease hair loss. One of the pathways explored is indeed the maintenance of the vascularization around the hair follicle.

### SUMMARY OF THE INVENTION

It has now surprisingly been found that 4-aminopiperidine compounds of specific formula (I), that will be defined below, exhibit, inter alia, a specific local activity on the degree of fibroblast contractility. By virtue of their effect on the tension of the fibroblasts and therefore on the static (isometric) tension of the connective tissue, they ensure a beneficial effect on the vascularization of the hair follicle, in particular in the processes of reimplantation of the follicle after each growth cycle. These 4-aminopiperidine compounds surprisingly have an activity that promotes improvement of the density of human keratin fibers. Thus, these compounds have a beneficial effect on the growth of human hair, but also on the growth of the eyelashes and of certain human body hairs.

The present invention therefore features administration, in particular topical cosmetic application, as agent for inducing and/or stimulating the growth of human keratin fibers, in particular the eyelashes and the hair, and/or preventing their loss and/or increasing their density, of the 4-aminopiperidine compounds of specific formula (I).

The expression “increasing the density of keratin fibers, and in particular hair density” means increasing the number of keratin fibers, in particular of hairs, per cm² of skin from where said fibers emerge, such as the scalp.

The present invention therefore features administration, in particular topical cosmetic application, as agent for inducing and/or stimulating the growth of human keratin fibers, in particular the eyelashes and the hair, and/or preventing their loss and/or increasing their density, of an effective amount of at least one 4-aminopiperidine compound of formula (I): 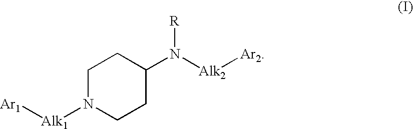
in which:

Alk₁ and Alk₂ are, independently of one another, a linear saturated C₁-C₁₀ or unsaturated C₂-C₁₀, or branched C₃-C₁₀ saturated or unsaturated, alkylene radical (divalent radical);

Ar₁ is a phenyl group optionally substituted with one or more radicals, which may be identical or different, selected from among —F, —CF3, —R1, —OR1 and —NR1R2;

Ar₂ is a phenyl group optionally substituted with one or more radicals, which may be identical or different, selected from among —F, —CF3 and —NR1R2;

R is a hydrogen atom or a saturated or unsaturated, C₁-C₁₀ linear or C₃-C₁₀ branched alkyl radical, optionally substituted with a group selected from among Ar₁, —OR₁ and —NR₁R₂;

R₁ and R₂ are each, independently of one another, a linear saturated C₁-C₇ or unsaturated C₂-C₇, or branched or cyclic C₃-C₇ saturated or unsaturated, alkyl radical;

and the salts, optical isomers and solvates thereof.

### DETAILED DESCRIPTION OF BEST MODE AND SPECIFIC/PREFERRED EMBODIMENTS OF THE INVENTION

In formula (I), the alkyl groups can in particular be selected, as appropriate, from among the groups: methyl, ethyl, n-propyl, isopropyl, n-butyl, isobutyl, tert-butyl, pentyl, hexyl, heptyl, octyl, nonyl and decyl. Similarly, the divalent alkylene groups can be selected from among methylene, ethylene, n-propylene, isopropylene, n-butylene, isobutylene, tert-butylene, pentylene, hexylene, heptylene, octylene, nonylene and decylene radicals.

For the compounds of formula (I), preferred are those having the following meanings:

Alk₁ and Alk₂ are each, independently of one another, a linear C₁-C₁₀ or branched C₃-C₁₀ saturated alkylene radical;

Ar₁ is a phenyl group optionally substituted with one or more radicals, which may be identical or different, selected from —R₁ and —OR₁;

Ar₂ is a phenyl group optionally substituted with one or more —CF₃ radicals;

R is a hydrogen atom or a linear C₁-C₄ or branched C₃-C₄, saturated alkyl radical, optionally substituted with a group selected from among —Ar₁, —OR₁ and —NR₁R₂;

R₁ and R₂ are each, independently of one another, a saturated linear C₁-C₄ alkyl radical.

Preferably, the compounds of formula (I) are employed in which:

Alk₁ and Alk₂ are each, independently of one another, a linear C₁-C₄ or branched C₃-C₄, saturated alkylene radical;

Ar₁ and Ar₂ are each a phenyl group;

R is a hydrogen atom or a linear C₁-C₄ or branched C₃-C₄, saturated alkyl radical, optionally substituted with a group selected from among —Ar₁, —OR₁ and —NR₁R₂;

R₁ and R₂ are each, independently of one another, a saturated linear C₁-C₄ alkyl radical.

More preferably, the compounds of formula (I) are employed in which:

Alk₁ and Alk₂ are each, independently of one another, a linear C₁-C₄ or branched C₃-C₄, saturated alkylene radical;

Ar₁ and Ar₂ are each a phenyl group;

R is a hydrogen atom or a linear C₁-C₄ or branched C₃-C₄, saturated alkyl radical, optionally substituted with a group selected from among a phenyl radical, —NR₁R₂, and preferably a hydrogen atom.

The acceptable salts of the compounds of the present invention comprise conventional non-toxic salts of said compounds, such as those formed from organic or inorganic acids. By way of example, representative are the salts of mineral acids, such as sulfuric acid, hydrochloric acid, hydrobromic acid, hydriodic acid, phosphoric acid or boric acid. Also exemplary are the salts of organic acids, which may contain one or more carboxylic, sulfonic or phosphonic acid groups. They may be linear, branched or cyclic aliphatic acids or alternatively aromatic acids. These acids may also contain one or more heteroatoms selected from O and N, for example in the form of hydroxyl groups. Particularly exemplary are propionic acid, acetic acid, terephthalic acid, citric acid and tartaric acid.

The preferred salts are those obtained from hydrochloric acid, sulfuric acid, acetic acid, tartaric acid or citric acid.

The acceptable solvates of the compounds of the present invention comprise conventional solvates such as those formed during the final step of preparation of said compounds due to the presence of solvents. By way of example, representative are the solvates due to the presence of water or of linear or branched alcohols such as ethanol or isopropanol.

Among the compounds of formula (I), exemplary are the Compounds 1 to 28 described below.

| Compound | Name | Ar₁ | Alk₁ | Alk₂ | Ar₂ | R |
|---|---|---|---|---|---|---|
| 1 | 1-(2-phenylethyl)-4- | Ph | CH₂CH₂ | CH₂CH₂CH₂ | Ph | H |
| | [N-(3-phenylpropyl)] | | | | | |
| | aminopiperidine | | | | | |
| 2 | 1-(2-phenylethyl)-4-(N- | Ph | CH₂CH₂ | CH₂ | Ph | H |
| | benzyl)amino-piperidine | | | | | |
| | (known compound) | | | | | |
| 3 | 1-(2-phenylethyl)-4- | Ph | CH₂CH₂ | CH₂ | (3,5)-CF₃-Ph | H |
| | [N-(3,5-bistrifluoro- | | | | | |
| | methyl)benzyl]aminopi- | | | | | |
| | peridine | | | | | |
| 4 | 1-(2-phenylethyl)-4- | Ph | CH₂CH₂ | CH₂CH₂ | Ph | H |
| | [N-(2-phenyl-ethyl)]amino- | | | | | |
| | piperidine | | | | | |
| 5 | 1-(2-phenylethyl)-4- | Ph | CH₂CH₂ | CHCH₃ | Ph | H |
| | [N-(1-phenyl-ethyl)]amino- | | | | | |
| | piperidine | | | | | |
| 6 | 1-benzyl-4-[N-(1-phenyl- | Ph | CH₂ | CHCH₃ | Ph | H |
| | ethyl)]amino-piperidine | | | | | |
| 7 | 1-[2-(3,4-dimethoxy)- | (3,4)-OMe-Ph | CH₂CH₂ | CH₂CH₂CH₂ | Ph | H |
| | phenylethyl]-4-[N-(3- | | | | | |
| | phenyl-propyl)]amino- | | | | | |
| | piperidine | | | | | |
| 8 | 1-(3-phenylpropyl)-4-[N- | Ph | CH₂CH₂CH₂ | CH₂CH₂CH₂ | Ph | H |
| | (3-phenyl-propyl)]amino- | | | | | |
| | piperidine | | | | | |
| 9 | 1-(2-phenylethyl)-4-[N- | Ph | CH₂CH₂ | CH₂CH₂CH₂CH₂ | Ph | H |
| | (4-phenyl-butyl)]amino- | | | | | |
| | piperidine | | | | | |
| 10 | 1-(2-phenylethyl)-4-[N- | Ph | CH₂CH₂ | CHCH₂CH₃ | Ph | H |
| | (1-phenyl-propyl)]amino- | | | | | |
| | piperidine | | | | | |
| 11 | 1-[2-(4-tert-butyl)- | 4-tBu-Ph | CH₂CH₂ | CH₂CH₂CH₂ | Ph | H |
| | phenyl-ethyl]-4-[N- | | | | | |
| | (3-phenylpropyl)]amino- | | | | | |
| | piperidine | | | | | |
| 12 | 1-(2-phenylethyl)-4-[N- | Ph | CH₂CH₂ | CHCH₃ (S) | Ph | H |
| | (1S)-1-phenylethyl]- | | | | | |
| | amino-piperidine | | | | | |
| 13 | 1-[2-(4-tert-butyl)phenyl- | 4-tBu-Ph | CH₂CH₂ | CHCH₃ | Ph | H |
| | ethyl]-4-[N-(1- | | | | | |
| | phenylethyl)]amino- | | | | | |
| | piperidine | | | | | |
| 14 | 1-(4-phenylbutyl)-4-[N- | Ph | CH₂CH₂CH₂CH₂ | CH₂CH₂CH₂ | Ph | H |
| | (3-phenyl propyl)]amino | | | | | |
| | piperidine | | | | | |
| 15 | 1-(3-phenylpropyl)-4-[N- | Ph | CH₂CH₂CH₂ | CHCH₂CH₃ | Ph | H |
| | (1-phenyl propyl)]amino | | | | | |
| | piperidine | | | | | |
| 16 | 1-(3-phenyl propyl)-4-[N- | Ph | CH₂CH₂CH₂ | CHCH₃ (S) | Ph | H |
| | (1S)-1-phenyl ethyl]amino | | | | | |
| | piperidine | | | | | |
| 17 | 1-(2-phenyl ethyl)-4- | Ph | CH₂CH₂ | CH₂CH₂CH₂ | Ph | Me |
| | [N-methyl-N- | | | | | |
| | (3-phenylpropyl)]amino | | | | | |
| | piperidine | | | | | |
| 18 | 1-(2-phenyl ethyl)-4-[N,N- | Ph | CH₂CH₂ | CH₂CH₂ | Ph | CH₂CH₂Ph |
| | di(2-phenyl ethyl)]amino | | | | | |
| | piperidine | | | | | |
| 19 | 1-(2-phenyl ethyl)-4- | Ph | CH₂CH₂ | CHCH₃ | Ph | Et |
| | [N-ethyl-N- | | | | | |
| | (1-phenylethyl)]amino | | | | | |
| | piperidine | | | | | |
| 20 | 1-(2-phenyl ethyl)-4- | Ph | CH₂CH₂ | CHCH₃ | Ph | CH₂CH₂CH₂Ph |
| | [N-(2-phenylpropyl)-N- | | | | | |
| | (1-phenyl ethyl)]amino | | | | | |
| | piperidine | | | | | |
| 21 | 1-(2-phenyl ethyl)-4- | Ph | CH₂CH₂ | CH₂CH₂CH₂ | Ph | CH₂CH₂Ph |
| | [N-(3-phenylpropyl)-N- | | | | | |
| | phenyl-ethyl]amino | | | | | |
| | piperidine | | | | | |
| 22 | 1-(2-phenyl ethyl)-4- | Ph | CH₂CH₂ | CH₂CH₂CH₂ | Ph | CH₂Ph |
| | [N-benzyl-N-(3- | | | | | |
| | phenylpropyl)]amino | | | | | |
| | piperidine | | | | | |
| 23 | 1-(2-phenyl ethyl)-4- | Ph | CH₂CH₂ | CH₂CH₂CH₂ | Ph | Et |
| | [N-ethyl-N-(3- | | | | | |
| | phenylpropyl)]amino | | | | | |
| | piperidine | | | | | |
| 24 | 1-(2-phenyl ethyl)-4- | Ph | CH₂CH2 | CH₂CH₂CH₂ | Ph | Pr |
| | [N-propyl-N-(3- | | | | | |
| | phenylpropyl)]amino | | | | | |
| | piperidine | | | | | |
| 25 | 1-(2-phenylethyl)-4- | Ph | CH₂CH₂ | CH₂CH₂CH₂ | Ph | n-Bu |
| | [N-butyl-N-3- | | | | | |
| | (3-phenyl propyl)]amino | | | | | |
| | piperidine | | | | | |
| 26 | 1-(2-phenylethyl)-4- | Ph | CH₂CH₂ | CH₂CH₂CH₂ | Ph | CH₂CH₂ |
| | [N-2-(3,4-dimethoxy) | | | | | Ph[(3,4)-OMe-] |
| | phenylethyl-N-(3-phenyl | | | | | |
| | propyl)]amino piperidine | | | | | |
| 27 | 1-(2-phenyl ethyl)-4-[N- | Ph | CH₂CH₂ | CH₂CH₂CH₂ | Ph | (CH₂)₂N(Me)₂ |
| | 2-dimethylamino-ethyl-N- | | | | | |
| | (3-phenylpropyl)]amino | | | | | |
| | piperidine | | | | | |
| 28 | 1-(2-phenylethyl)-4- | Ph | CH₂CH₂ | CH₂CH₂CH₂ | Ph | CH₂CH₂OCH₃ |
| | [N-2-methoxy ethyl-N- | | | | | |
| | (3-phenylpropyl)]amino | | | | | |
| | piperidine | | | | | |

The compounds (I) that are particularly preferred are Compounds 1, 5, 6, 21, 25 and 27, and in particular 1 and 5.

In general, the compounds of formula (I) for which R is a hydrogen atom can be prepared according to schemes I and II hereinafter, by alkylation of 1,1-dimethyl-1,1-dimethyl-4-oxopiperidinium iodide with an alkylamine (A), especially at a temperature of from 40° C. to 100° C., in particular in the presence of a mineral base (for example, sodium bicarbonate or sodium hydroxide) in a water/ethanol mixture, to form a piperidone (B) which is isolated and then purified, for example, by silica gel chromatography.

The piperidone (B) obtained is subsequently alkylated and reduced with an alkylamine (C), especially in the presence of acetic acid (1 molar equivalent) and of NaBH(OAc)₃ (sodium triacetoxyborohydride) (2 molar equivalents), in particular in dichloromethane, and especially at ambient temperature (25° C.).

The reaction medium is subsequently extracted 3 times with water at acidic pH. The aqueous phases are subsequently combined and extracted 3 times with dichloromethane. The organic phases are combined, dried and concentrated to obtain the expected product (la), it being possible for the latter to be optionally purified on silica gel and/or by precipitation.

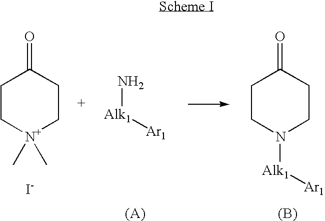

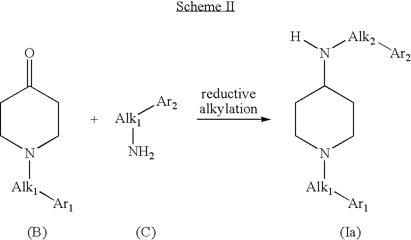

Compounds 1 to 16 were synthesized according to the general process of scheme II.

The compounds of formula (I) for which R is other than a hydrogen atom can be prepared according to scheme III described hereinafter.

The compound (la) is acylated with acid chloride of formula RCOCI in the presence of an organic base (for example, pyridine or triethylamine) to produce an intermediate amide which is reduced in the presence of LiAlH₄ (lithium aluminum hydride), especially in an ethereal solvent (for example dioxane, diethyl ether or tetrahydrofuran), especially by heating to a temperature of from 40° C. and 100° C., and thus makes it possible to obtain the compound (lb)

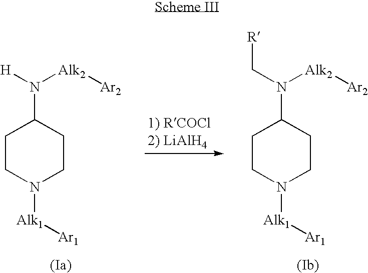

Compounds 17 to 28 were synthesized according to this general process of scheme III.

The present invention also features formulating at least one 4-aminopiperidine compound of formula (I) or of a salt, optical isomer or solvate thereof, into cosmetic care and/or makeup compositions for human keratin fibers, useful for reducing the loss of keratin fibers and/or increasing their density.

This invention also features formulating at least one 4-aminopiperidine compound of formula (I) or of a salt, optical isomer or solvate thereof, into care and/or treatment compositions for human keratin fibers, useful to induce and/or stimulate the growth of keratin fibers and/or prevent their loss and/or increase their density.

The human keratin fibers to which the invention relates are in particular the hair, the eyebrows, the eyelashes, beard hairs, moustache hairs and pubic hairs. More especially, the present invention relates to human hair and/or eyelashes.

Thus, the present invention also features formulating at least one 4-aminopiperidine compound of formula (I) or of a salt, optical isomer or solvate thereof, into cosmetic hair care compositions for human beings, for treating (reducing) hair loss and/or increasing hair density and/or treating androchronogenetic alopecia.

This invention also features formulating at least one 4-aminopiperidine compound of formula (I) or of a salt, optical isomer or solvate thereof, into hair compositions useful to induce and/or stimulate the growth of human hair and/or prevent its loss and/or increase its density and/or treat androgenic alopecia. In particular, these compositions make it possible to maintain the head of hair in a good condition and/or to combat natural hair loss, more particularly in men.

The present invention also features formulating at least one 4-aminopiperidine compound of formula (I) or of a salt, optical isomer or solvate thereof, into cosmetic hair care compositions for human beings, for treating alopecia of natural origin, and in particular androgenic alopecia.

This invention also features formulating at least one 4-aminopiperidine compound of formula (I) or of a salt, optical isomer or solvate thereof, into hair care compositions for treating alopecia of natural origin, and in particular androgenic alopecia.

This invention also features formulating at least one 4-aminopiperidine compound of formula (I) or of a salt, optical isomer or solvate thereof, into cosmetic care and/or makeup compositions for the eyelashes of human beings, for inducing and/or stimulating the growth of the eyelashes and/or increasing their density, and also formulating at least one compound of formula (I) or of a salt, optical isomer or solvate thereof, into care and/or treatment compositions for the eyelashes of human beings, suited to induce and/or stimulate the growth of the eyelashes and/or increase their density. These compositions thus make it possible to maintain the eyelashes in a good condition and/or to improve their condition and/or their appearance.

The present invention also features formulating at least one 4-aminopiperidine compound of formula (I) or of a salt, optical isomer or solvate with an acid thereof, into compositions for use in treating disorders associated with a reduction in cutaneous capillary circulation or vascularization, and in particular of the hair follicle, in human beings.

The effective amount of a 4-aminopiperidine compound of formula (I) or of a salt, optical isomer or solvate thereof corresponds to the amount required in order to obtain the desired result (in particular, i.e., a regime or regimen to increase the density of keratin fibers or promote their growth). Those skilled in the art are therefore in a position to evaluate this effective amount, which depends on the nature of the amine used, on the individual to whom it is applied, and on the period of time of this application.

In the subsequent text, and unless otherwise indicated, the amounts of the various ingredients of the composition are given as percentage by weight relative to the total weight of the composition.

To provide an order of magnitude, according to the invention, the 4-aminopiperidine compound of formula (I) or a salt, optical isomer or solvate thereof can be used in an amount advantageously representing from 10⁻³% to 10% of the total weight of the composition, and preferably in an amount representing from 10⁻²% to 5% of the total amount of the composition, for example from 0.5% to 2%.

The compositions of the invention may be for cosmetic or pharmaceutical (in particular dermo-pharmaceutical) application. Preferably, the compositions of the invention are for cosmetic application. Thus, the composition should contain a non-toxic physiologically acceptable medium that can be applied to the skin, including the scalp and the eyelids, and to keratin fibers such as the hair and the eyelashes. For the purpose of the invention, the term “cosmetic” means a composition that has a pleasant appearance, odor and feel.

The 4-aminopiperidine compounds of formula (I) or the salts, optical isomers and solvates thereof can be formulated into compositions to be ingested, injected or applied to the skin or keratin fibers (to any area of the skin or fibers to be treated).

The 4-aminopiperidine compounds of formula (I) or the salts, optical isomers and solvates can be administered orally in an amount of from 0.1 to 300 mg per day, 5 to 10 mg/d.

A preferred composition of the invention is a composition for cosmetic use, in particular for topical application to the skin and keratin fibers, and more especially to the scalp, the hair and the eyelashes.

This composition may be in any of the known galenic forms suitable for the method of administration.

For topical application to the skin and keratin fibers, including the scalp, the composition may be in the form of an aqueous, alcoholic, aqueous-alcoholic or oily solution or suspension, an emulsion or dispersion of more or less fluid, and in particular liquid or semi-liquid, consistency, obtained by dispersion of a fatty phase in an aqueous phase (ONV) or inversely (W/O), a solid dispersion or emulsion (ONV) or (W/O), an aqueous, aqueous-alcoholic or oily gel that is more or less fluid or solid, a loose or compacted powder to be used as it is or to be incorporated into a physiologically acceptable medium, or else microcapsules or microparticles, or vesicular dispersions of ionic and/or non-ionic type.

Also intended are compositions in the form of a mousse or alternatively in the form of a spray or of an aerosol, which then comprise a pressurized propellant.

The composition can thus be in the form of a lotion, serum, milk, O/W or W/O cream, gel, salve, ointment, powder, balm, patch, soaked pad, soap, bar or mousse.

In particular, the composition to be applied to the scalp or the hair can be in the form of a hair care lotion, for example for daily or twice-weekly application, a shampoo or a hair conditioner, in particular for twice-weekly or weekly application, a liquid or solid scalp cleansing soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for application to the eyelashes or body hairs, the composition to which the invention relates may be in the form of a pigmented or unpigmented mascara, to be applied with a brush to the eyelashes or alternatively to beard or moustache hair.

For a composition for injection, the composition may be in the form of an aqueous lotion or an oily suspension, for example in the form of a serum. For oral administration, the composition may be in the form of capsules, granules, oral syrups or tablets.

According to a specific embodiment, the composition according to the invention is in the form of a hair cream or hair lotion, a shampoo, a hair conditioner or a mascara for the hair or for the eyelashes.

The amounts of the various constituents of the physiological medium of the composition according to the invention are those generally used in the fields under consideration. In addition, these compositions are prepared according to the usual methods.

When the composition is an emulsion, the proportion of the fatty phase may range from 2% to 80% by weight, and preferably from 5% to 50% by weight, relative to the total weight of the composition. The aqueous phase is adjusted as a function of the content of fatty phase and of compound(s) (I) and also of that of the optional additional ingredients, to obtain 100% by weight. In practice, the aqueous phase is from 5% to 99.9% by weight.

The fatty phase may contain fatty or oily compounds that are liquid at ambient temperature (25° C.) and atmospheric pressure (760 mmHg), which are generally known as oils. These oils may be mutually compatible or incompatible and may form a macroscopically homogeneous liquid fatty phase or a two-phase or three-phase system. In addition to these oils, the fatty phase may contain waxes, gums, lipophilic polymers or “pasty” or viscous products containing solid parts and liquid parts.

The aqueous phase contains water and, optionally, an ingredient that is miscible in all proportions with water, for instance C₁ to C₈ lower alcohols such as ethanol or isopropanol, polyols such as propylene glycol, glycerol or sorbitol, or alternatively acetone or ether.

The emulsifiers and coemulsifiers used to obtain a composition in the form of an emulsion are those generally used in the cosmetics and pharmaceutical fields. Their nature also depends on the sense of the emulsion. In practice, the emulsifier and, optionally, the coemulsifier are present, in the composition, in a proportion ranging from 0.1% to 30% by weight, preferably from 0.5% to 20% by weight, and better still from 1% to 8%. The emulsion can also contain lipid vesicles, and in particular liposomes.

When the composition is in the form of an oily solution or gel, the fatty phase may represent more than 90% of the total weight of the composition.

Advantageously, for a hair application, the composition is an aqueous, alcoholic or aqueous-alcoholic solution or suspension, and better still a water/ethanol solution or suspension. The alcoholic fraction may represent from 5% to 99.9%, and better still from 8% to 80%.

For a mascara application, the composition is in particular a wax-in-water or wax-in-oil dispersion, a gelled oil or an aqueous gel. It may be pigmented or unpigmented.

The compositions of the invention may also comprise other ingredients that are normally used in the fields under consideration, selected from among aqueous-phase or oily-phase solvents, thickeners or gelling agents, dyestuffs that are soluble in the medium of the composition, solid particles of the filler or pigment type, antioxidants, preservatives, fragrances, electrolytes, neutralizing agents, UV blockers, for example sunscreens, film-forming polymers, cosmetic and pharmaceutical active agents with a beneficial effect on the skin or keratin fibers, other than the compounds of formula (I) (such as vitamins), and mixtures thereof. These additives may be present in the composition according to the amounts generally used in the cosmetics and dermatological field, and in particular in a proportion of from 0.01 % to 50% of the total weight of the composition, and better still from 0.1 % to 20%, and for example from 0.1 % to 10%. Depending on their nature, these adjuvants may be introduced into the fatty phase, into the aqueous phase and/or into the lipid vesicles, and in particular liposomes.

Of course, those skilled in the art will take care to select the optional additional ingredients and/or the amounts thereof in such a way that the advantageous properties of the compositions according to the invention, in particular i.e., the increase in density of the keratin fibers, are not, or are not substantially, adversely affected by the envisaged addition.

As solvents according to the invention, exemplary are C₂ to C₈ lower alcohols, such as ethanol, isopropanol, propylene glycol and certain light cosmetic oils, such as C₆ to C₁₆ alkanes.

As oils according to the invention, exemplary are oils of mineral origin (liquid petroleum jelly or hydrogenated isoparaffin), oils of plant origin (liquid fraction of shea butter, sunflower oil, apricot oil, soybean oil, fatty alcohol or fatty acid), oils of animal origin (perhydrosqualene), synthetic oils (fatty acid esters, Purcellin oil), silicone oils (linear or cyclic polydimethylsiloxanes, phenyl trimethicones) and fluoro oils (perfluoropolyethers). As waxes, exemplary are silicone waxes, beeswaxes, candelilla wax, rice wax, carnauba wax, paraffin wax or polyethylene wax.

As emulsifiers according to the invention, exemplary are glycerol stearate, glycerol laurate, sorbital stearate, sorbitol oleate, alkyl dimethicone copolyols (with alkyl≧8) and mixtures thereof for a W/O emulsion. Polyethylene glycol monostearate or monolaurate, polyoxyethylenated sorbitol stearate or oleate, and dimethicone copolyols, and mixtures thereof, may also be used for an O/W emulsion. The emulsifier and the coemulsifier are present, in the composition, in a proportion ranging from 0.3% to 30% by weight, and preferably from 0.5% to 20% by weight relative to the total weight of the composition.

As hydrophilic gelling agents according to the invention, exemplary are carboxyvinyl polymers (carbomer), acrylic copolymers such as acrylate/alkyl acrylate copolymers, polyacrylamides, polysaccharides such as hydroxypropylcellulose, natural gums and clays, and as lipophilic gelling agents, exemplary are modified clays such as bentones, metal salts of fatty acids, for instance aluminum stearates, hydrophobic-treated silica and ethylcellulose, and mixtures thereof.

As cosmetic or pharmaceutical active agent other than the amines of formula (I), the compositions may contain an additional hydrophilic active agent selected from among proteins or protein hydrolysates, amino acids, polyols, urea, allantoin, sugars and sugar derivatives, water-soluble vitamins, plant extracts (those from Iridacea plants or from soybean) and hydroxy acids (fruit acid or salicylic acid); and/or an additional lipophilic active agent selected from retinol (vitamin A) and its derivatives, especially an ester (retinyl palmitate), tocopherol (vitamin E) and its derivatives, especially an ester (tocopheryl acetate or palmitate), essential fatty acids, ceramides, essential oils, salicylic acid derivatives such as 5-n-octanoylsalicylic acid, hydroxy acid esters, and phospholipids such as lecithin, and mixtures thereof.

According to a specific embodiment of the invention, the ((dialkylamino)alkoxy)ethanol ester of formula (I) or a salt thereof may be combined with at least one additional compound that promotes the regrowth and/or limits the loss of keratin fibers (hair or eyelashes). These additional compounds are in particular selected from the lipoxygenase inhibitors as described in EP-0,648,488, the bradykinin inhibitors described in particular in EP-0,845,700, prostaglandins and derivatives thereof, in particular those described in WO 98/33497, WO 95/11003, JP 97-100091 and JP 96-134242, prostaglandin receptor agonists and antagonists, the non-prostanoic prostaglandin analogues as described in EP-1,175,891, EP-1,175,890, WO 01/74307, WO 01/74313, WO 01/74314, WO 01/74315 or WO 01/72268, and mixtures thereof.

As other additional compounds that promote the growth of the hair that may be present in the compositions according to the invention, exemplary are 15-hydroxyprostaglandin dehydrogenase inhibitors such as those described in WO 03/090699, WO 04/028441, WO 04/039306, WO 04/047776, WO 04/069213 or EP-1,505,576.

As other additional agents that promote the growth of the hair that may be present in the composition according to the invention, exemplary are vasodilators, anti-androgens, cyclosporins and analogues thereof, anti-microbial and anti-fungal agents, anti-inflammatories and retinoids, alone or in a mixture.

The vasodilators that can be used are in particular potassium-channel agonists, including minoxidil, and also the compounds described in U.S. Pat. Nos. 3,382,247, 5,756,092, 5,772,990, 5,760,043, 5,466,694, 5,438,058 and 4,973,474, cromakalim, nicorandil and diaxozide, alone or in combination.

The anti-androgens that can be used include, in particular, steroidal or non-steroidal 5α-reductase inhibitors, for instance finasteride and the compounds described in U.S. Pat. No. 5,516,779, cyprosterone acetate, azelaic acid and the salts and derivatives thereof, and the compounds described in U.S. Pat. No. 5,480,913, flutamide, oxendolone, spironolactone, diethylstilbestrol and the compounds described in U.S. Pat. Nos. 5,411,981, 5, 565,467 and 4,910,226.

The anti-microbial or anti-fungal compounds can be selected from among selenium derivatives, octopirox, triclocarban, triclosan, zinc pyrithione, itraconazole, asiatic acid, hinokitiol, mipirocine, tetracyclines, in particular erythromycin and the compounds described in EP-0,680,745, clinycin hydrochloride, benzoyl peroxide or benzyl peroxide, minocyclin and compounds belonging to the imidazole class, such as econazole, ketoconazole or miconazole or salts thereof, nicotinic acid esters, including in particular tocopheryl nicotinate, benzyl nicotinate and C₁-C₆ alkyl nicotinates, for instance methyl nicotinate or hexyl nicotinate.

The anti-inflammatories can be selected from among steroidal anti-inflammatories such as glucocorticoids, corticosteroids (for example: hydrocortisone) and non-steroidal anti-inflammatories such as glycyrrhetinic and α-bisabolol, benzydamine, salicylic acid and the compounds described in EP-0,770,399, WO 94/06434 and FR 2,268,523.

The retinoids can be selected from among isotretinoin, acitretin and tazarotene.

As other additional active compounds for promoting the growth and/or limiting the loss of the hair that can be used in combination with the compounds of formula (I), which may or may not be salified, exemplary are aminexil, 6-0-[(9Z,12Z)octadeca-9,12-dienoyl]hexapyranose, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic or acylhexosaccharidic acids, substituted arylethylenes, N-acylamino acids, flavonoids, ascomycin derivatives and analogues, histamine antagonists, saponins, proteoglycanase inhibitors, oestrogen agonists and antagonists, pseudoterines, cytokines and growth factor promoters, inhibitors of IL-1 or of IL-6, IL-10 promoters, TNF inhibitors, benzophenones and hydantoin, retinoic acid; vitamins, for instance vitamin D, vitamin B12 analogues and pantothenol; triterpenes such as ursolic acid and the compounds described in U.S. Pat. Nos. 5,529,769, 5,468,888 and 5,631,282; anti-pruriginous agents, for instance thenaldine, trimeprazine or cyproheptadine; anti-parasitic agents, in particular metronidazole, crotamiton or pyrethrinoids; calcium antagonists, for instance cinnarizine, diltiazem, nimodipine, verapamil, alverine and nifedipine; hormones such as oestriol or analogues thereof, thyroxine and salts thereof, progesterone; FP receptor (type F prostaglandin receptor) antagonists such as latanoprost, bimatoprost, travoprost or unoprostone; and mixtures thereof.

The compositions comprising at least one ((dialkylamino)alkoxy)ethanol ether of formula (I), which may or may not be in salified form, may be in liposomal form, as described in particular in WO 94/22468. Thus, the compound encapsulated in the liposomes may be delivered selectively to the hair follicle.

The compositions to which the invention relates can be applied to the alopecic areas of the scalp and the hair of an individual, and optionally left in contact for several hours and optionally rinsed off.

The compositions containing an effective amount of the ((dialkylamino)alkoxy)ethanol ether of formula (I), which may or may not be in salified form, may, for example, be applied in the evening, kept in contact throughout the night and optionally shampooed out in the morning. These applications may be repeated daily for one or more months according to the individual.

Thus, the present invention also features a cosmetic treatment regime or regimen for human keratin fibers and/or the skin from where these fibers emerge, including the scalp and the eyelids, to stimulate the growth of human keratin fibers such as the hair and the eyelashes of human beings and/or to prevent their loss, which comprises applying, to the human keratin fibers and/or the skin from where the fibers emerge, a cosmetic composition comprising an effective amount of at least one 4-aminopiperidine compound of formula (I) or a salt, optical isomer or solvate thereof, in leaving said composition in contact with the keratin fibers and/or the skin, and optionally in rinsing the keratin fibers and/or the skin.

This treatment has the characteristics of a cosmetic process insofar as it makes it possible to improve the aesthetics of the keratin fibers, and in particular of the hair and the eyelashes, by giving them greater vigor and an improved appearance. In addition, it can be used daily for several months, without medical prescription.

More especially, the present invention features a cosmetic care regime or regimen for human hair and/or the human scalp, for the purpose of improving their condition and/or their appearance, which comprises applying, to the hair and/or the scalp, a cosmetic composition comprising at least one 4-aminopiperidine compound of formula (I) or a salt, optical isomer or solvate thereof, in leaving said composition in contact with the hair and/or the scalp, and optionally in rinsing the hair and/or the scalp.

This invention also features a cosmetic care and/or makeup regime or regimen for human eyelashes, for the purpose of improving their condition and/or their appearance, which comprises applying a mascara composition comprising at least one 4-aminopiperidine compound of formula (I) or a salt, optical isomer or solvate thereof, and in leaving said composition in contact with the eyelashes. This mascara composition can be applied alone or as an undercoat for a conventional pigmented mascara and can be removed like a conventional pigmented mascara.

Advantageously, in the treatment according to the invention, from 5 to 500 μl of a solution or composition as defined above, comprising from 0.001% to 5% of compound of formula (I), are applied to the areas of the scalp to be treated.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Synthesis of 1-(2-phenylethyl)-4-[N-(3-phenylpropyl)]aminopiperidine (Compound 1)

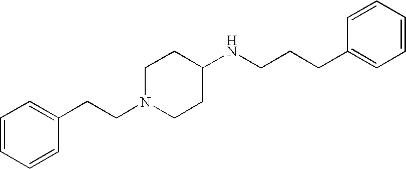

10 g of 1-phenylethyl-4-piperidone (1 molar equivalent) and 9.2 ml of 3-phenylpropylamine (1.3 molar equivalents) were introduced into 100 ml of dichloromethane in the presence of 3.09 ml of acetic acid (1 molar equivalent) and 21.94 g of NaBH(OAc)₃ (2 molar equivalents), and the mixture was left to react at ambient temperature (25° C.) for 20 hours. After treatment and purification, 15.8 g of product were obtained in the form of a brown oil (99% yield). The mass spectrum confirms the expected structure.

### Example 2

### Synthesis of 1-(2-phenylethyl)-4-[N-ethyl-N-3-(phenylpropyl)]aminopiperidine (Compound 23)

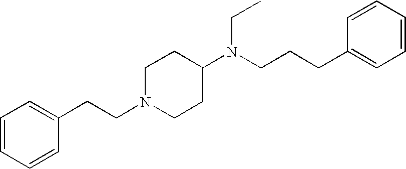

15.8 g of 1-(2-phenylethyl)-4-[N-(3-phenylpropyl)]aminopiperidine (obtained according to Example 1) and 3.65 ml of acetyl chloride (1.05 molar equivalents) were introduced into dichloromethane in the presence of 14.45 ml of triethylamine (2.1 molar equivalents) and the mixture was left to react at ambient temperature (25° C.) for 15 hours.

After treatment and purification over sintered silica, 15.5 g of 1-(2-phenylethyl)-4-N-[acetyl-N-3-(phenylpropyl)]aminopiperidine were obtained (87% yield).

15.5 g of the intermediate compound obtained were subsequently mixed with 8.5 g (5 molar equivalents) of LiAlH₄ in ethyl ether at reflux for 2 hours. After treatment and purification over sintered silica, 10.5 g of the expected product were obtained (70% yield).

### Example 3

### Demonstration of the dermodecontracting Effect of the 4-aminopiperidine Compounds According to the Invention

Principle of the Test:

The principle of this test entails studying the effect of the test product on a model of dermal equivalent consisting of a collagen matrix seeded with normal human fibroblasts.

These conditions are suited to mimic, in vitro, the dermal contractile phenomena that occur during facial expressions. Under these conditions, in fact, the cells spontaneously express tensile forces which induce a retraction of the collagen gel. This results in a decrease in the total surface area of the dermal equivalent over time. The measurement of this surface area makes it possible to evaluate the relaxing effects of the substances brought into contact beforehand with the dermal equivalent.

b) Protocol:

Two series of attached dermal equivalents containing normal human fibroblasts are prepared: a control series without any treatment, and a series treated with the test compound (1 μM). The experiment is repeated three times.

The dermal equivalents are prepared as described in Asselineau et al., Exp. Cell. Res., 1985,159, 536-539; Models in Dermatology, 1987, vol. 3 pp 1-7, in the following proportions:

| | |
|---|---|
| MEM medium (1.76×) with or without compound | 45% |
| Foetal calf serum: | 10% |
| NaOH (0.1N): | 5% |
| Acetic acid (1/1000): | 4% |
| Collagen: | 26% |
| Fibroblasts: | 11% |

The treated dermal equivalent differs from the control dermal equivalent in that 1 μM of the test compound is added thereto.

The collagen used is collagen type I (commercial solution). It is extracted from rat tail or from calf skin by acid hydrolysis and stored in an acidic medium at +4° C.; it polymerizes naturally by heating at 37° C. and by decreasing the degree of acidity. The collagen is dialyzed beforehand against successive baths of water+acetic acid.

The protocol is as follows: introduced into a 50 ml centrifuge tube kept in crushed ice are the 1.76×MEM medium in the presence of additives (1% glutamine,1% non-essential amino acids,1% sodium pyruvate, 1% fungizone and 1% penicillin/streptomycin), the foetal calf serum and the 0.1 N sodium hydroxide NaOH. The fibroblasts isolated from human skin explants are then added at the concentration of 1.5×10⁵ cells per 1 ml of culture medium.

A volume/volume mixture of collagen in acetic acid at 1/1000 is then added slowly, against the wall of the tube so as to observe the appearance of a whitish cloud.

The whole is then mixed carefully and dispensed into the wells of a 12-well culture plate (type Costar reference 3512) at a rate of 2 ml of mixture per well. The final cell concentration is 3×10⁴ cells/dermal equivalent, with a final collagen concentration of 1 mg/ml. The culture plate is then placed in an incubator at 37° C. with 5% CO₂.

Once formed after polymerization of the collagen, the dermal equivalents are left adherent to the culture support for 3 days and then detached from the support so that the contraction can begin. These attached dermal equivalents are taken out of the incubator in order to capture images for the purpose of measuring their surface area, this being done for each timepoint of the contraction kinetics (0, 4, 8 and 24 hours). They are immediately returned to the incubator from each measurement point.

The evaluation of the spontaneous contraction of the treated (with the test compound) and control (without test compound) dermal equivalents is carried out by measuring their surface area at various times after the beginning of the spontaneous contraction.

For this, a digital image is acquired for each treated or non-treated dermal equivalent by means of a camera (Camera CCD-Iris Sony DXC-107P) and the surface area is then calculated on each image by means of an image analysis system (Zeiss Axiovision 3.0). Corresponding to each surface area measurement is a percentage contraction equal to the ratio of the surface areas according to the formula:
% contraction=(Sp−Si)/Sp×100
where ‘Sp’ is the surface area of a well of the culture plate; it corresponds to the total surface area of the dermal equivalent before contraction, ‘Si’ is the surface area of the dermal equivalent at the instant i of the contraction kinetics.

c) Results:

### 1-(2-Phenylethyl)-4-[N-(3-phenylpropyl)]aminopiperidine (Compound No. 1) of formula

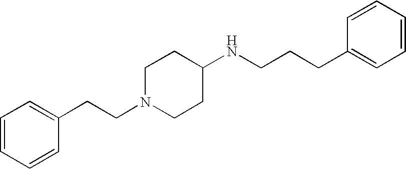 reduces the fibroblast contraction by 21% on average over the duration of the experiment (tested at 1 μM), compared with the control.

### 1-(2-Phenylethyl)-4-[N-(1 -phenylethyl)]aminopiperidine (Compound No. 5) of formula

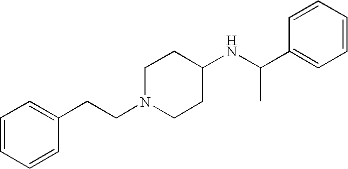 reduces the fibroblast contraction by 40% on average over the duration of the experiment (tested at 10 μM), compared with the control.

The 2 compounds tested therefore have a significant dermodecontracting effect and may therefore provide a beneficial effect on the vascularization of the hair follicle.

### Example 4

### Hair Lotion

| | | |
|---|---|---|
| Compound No. 1 | 1.00 | g |
| Propylene glycol | 30.00 | g |
| Ethyl alcohol | 40.00 | g |
| Water | qs 100.00 | g |

This lotion is applied to the scalp once or twice a day, for a few months, at a rate of 1 ml per application, massaging the scalp slightly so as to cause the active agent to penetrate. The head of hair is then dried in the open air. This lotion makes it possible to decrease hair loss and/or to promote hair regrowth and/or improve the appearance of the hair.

In this composition, Compound No. 1 can be replaced with Compound No. 2 or No. 3.

### Example 5

### Wax/Water Mascara

| | | |
|---|---|---|
| Beeswax | 6.00 | g |
| Paraffin wax | 13.00 | g |
| Hydrogenated jojoba oil | 2.00 | g |
| Water-soluble film-forming polymer | 3.00 | g |
| Triethanolamine stearate | 8.00 | g |
| Compound No. 1 | 1.00 | g |
| Black pigment | 5.00 | g |
| Preservative | qs | |
| Water | qsp 100 | g |

This mascara is applied to the eyelashes like a conventional mascara, with a mascara brush. It makes it possible to improve the appearance of the eyelashes.

In this composition, Compound No. 1 can be replaced with Compound No. 5 or No. 23.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for inducing and/or stimulating the growth of human keratin fibers and/or preventing their loss and/or increasing their density, comprising administering to an individual in need of such treatment, a thus effective amount of at least one 4-aminopiperidine compound of formula (I): 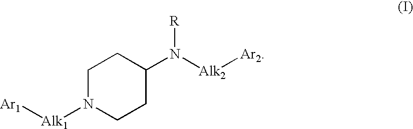
in which:
Alk₁ and Alk₂ are each, independently of one another, a linear saturated C₁-C₁₀ or unsaturated C₂-C₁₀, or branched C₃-C₁₀ saturated or unsaturated, alkylene radical (divalent radical);
Ar₁ is a phenyl group optionally substituted with one or more radicals, which may be identical or different, selected from among —F, —CF3, —R1, —OR1 and —NR1R2;
Ar₂ is a phenyl group optionally substituted with one or more radicals, which may be identical or different, selected from among —F, —CF3 and —NR1 R2;
R is a hydrogen atom or a saturated or unsaturated, C₁-C₁₀ linear or C₃-C₁₀ branched alkyl radical, optionally substituted with a group selected from among Ar₁, —OR₁ and —NR₁R₂;
R₁ and R₂ are each, independently of one another, a linear saturated C₁-C₇ or unsaturated C₂-C₇, or branched or cyclic C₃-C₇ saturated or unsaturated, alkyl radical;
or a salt, optical isomer or solvate thereof.

2. A topically applicable care and/or treatment composition for keratin fibers for human beings, useful to induce and/or stimulate the growth of keratin fibers and/or prevent their loss and/or increase their density, comprising a thus effective amount of at least one 4-aminopiperidine compound of formula (I): 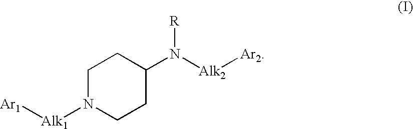
in which:
Alk₁ and Alk₂ are each, independently of one another, a linear saturated C₁-C₁₀ or unsaturated C₂-C₁₀, or branched C₃-C₁₀ saturated or unsaturated, alkylene radical (divalent radical);
Ar₁ is a phenyl group optionally substituted with one or more radicals, which may be identical or different, selected from among —F, —CF3, —R1, —OR1 and —NR1R2;
Ar₂ is a phenyl group optionally substituted with one or more radicals, which may be identical or different, selected from among —F, —CF3 and —NR₁R2;
R is a hydrogen atom or a saturated or unsaturated, C₁-C₁₀ linear or C₃-C₁₀ branched alkyl radical, optionally substituted with a group selected from among Ar₁, —OR₁ and —NR₁R₂;
R₁ and R₂ are each, independently of one another, a linear saturated C₁-C₇ or unsaturated C₂-C₇, or branched or cyclic C₃-C₇ saturated or unsaturated, alkyl radical;
or a salt, optical isomer or solvate thereof, formulated into a topically applicable, physiologically acceptable medium therefor.

3. The regime or regimen as defined by claim 1, said keratin fibers comprising hair, eyebrows, eyelashes, beard hairs, moustache hairs and/or pubic hairs.

4. A regime or regimen for reducing natural hair loss and/or increasing hair density and/or treating androchronogenetic alopecia, comprising administering to an individual in need of such treatment, a thus effective amount of at least one 4-aminopiperidine compound of formula (I): 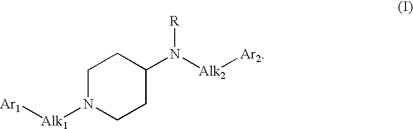
in which:
Alk₁ and Alk₂ are each, independently of one another, a linear saturated C₁-C₁₀ or unsaturated C₂-C₁₀, or branched C₃-C₁₀ saturated or unsaturated, alkylene radical (divalent radical);
Ar₁ is a phenyl group optionally substituted with one or more radicals, which may be identical or different, selected from among —F, —CF3, —R1, —OR1 and —NR1R2;
Ar₂ is a phenyl group optionally substituted with one or more radicals, which may be identical or different, selected from among —F, —CF3 and —NR1R2;
R is a hydrogen atom or a saturated or unsaturated, C₁-C₁₀ linear or C₃-C₁₀ branched alkyl radical, optionally substituted with a group selected from among Ar₁, —OR₁ and —NR₁R₂;
R₁ and R₂ are each, independently of one another, a linear saturated C₁-C₇ or unsaturated C₂-C₇, or branched or cyclic C₃-C₇ saturated or unsaturated, alkyl radical;
or a salt, optical isomer or solvate thereof.

5. A regime or regimen for the cosmetic care and/or makeup of the eyelashes of a human being, for inducing and/or stimulating the growth of the eyelashes and/or increasing their density, comprising topically onto the eyelashes of an individual in need of such treatment, a thus effective amount of at least one 4-aminopiperidine compound of formula (I): 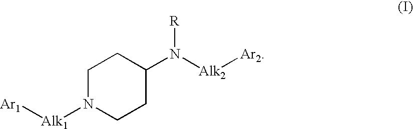
in which:
Alk₁ and Alk₂ are each, independently of one another, a linear saturated C₁-C₁₀ or unsaturated C₂-C₁₀, or branched C₃-C₁₀ saturated or unsaturated, alkylene radical (divalent radical);
Ar₁ is a phenyl group optionally substituted with one or more radicals, which may be identical or different, selected from among —F, —CF3, —R1, —OR1 and —NR1R2;
Ar₂ is a phenyl group optionally substituted with one or more radicals, which may be identical or different, selected from among —F, —CF3 and —NR1R2;
R is a hydrogen atom or a saturated or unsaturated, C₁-C₁₀ linear or C₃-C₁₀ branched alkyl radical, optionally substituted with a group selected from among Ar₁, —OR₁ and —NR₁R₂;
R₁ and R₂ are each, independently of one another, a linear saturated C₁-C₇ or unsaturated C₂-C₇, or branched or cyclic C₃-C₇ saturated or unsaturated, alkyl radical;
or a salt, optical isomer or solvate thereof.

6. A regime or regimen for treating a disorder associated with a reduction in cutaneous capillary circulation or vascularization in a human being, comprising administering to an individual in need of such treatment, a thus effective amount of at least one 4-aminopiperidine compound of formula (I): 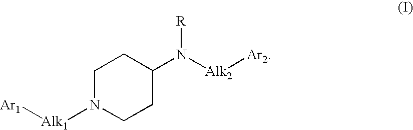
in which:
Alk₁ and Alk₂ are each, independently of one another, a linear saturated C₁-C₁₀ or unsaturated C₂-C₁₀, or branched C₃-C₁₀ saturated or unsaturated, alkylene radical (divalent radical);
Ar₁ is a phenyl group optionally substituted with one or more radicals, which may be identical or different, selected from among —F, —CF3, —R1, —OR1 and —NR1R2;
Ar₂ is a phenyl group optionally substituted with one or more radicals, which may be identical or different, selected from among —F, —CF3 and —NR1R2;
R is a hydrogen atom or a saturated or unsaturated, C₁-C₁₀ linear or C₃-C₁₀ branched alkyl radical, optionally substituted with a group selected from among Ar₁, —OR₁ and —NR₁R₂;
R₁ and R₂ are each, independently of one another, a linear saturated C₁-C₇ or unsaturated C₂-C₇, or branched or cyclic C₃-C₇ saturated or unsaturated, alkyl radical;
or a salt, optical isomer or solvate thereof.

7. The regime or regimen as defined by claim 1, wherein formula (I):
Alk₁ and Alk₂ are each, independently of one another, a linear C₁-C₁₀ or branched C₃-C₁₀, saturated alkylene radical;
Ar₁ is a phenyl group optionally substituted with one or more radicals, which may be identical or different, selected from —R₁ and —OR₁;
Ar₂ is a phenyl group optionally substituted with one or more —CF₃ radicals;
R is a hydrogen atom or a linear C₁-C₄ or branched C₃-C₄, saturated alkyl radical, optionally substituted with a group selected from among —Ar₁, —OR₁ and —NR₁R₂;
R₁ and R₂ are each, independently of one another, a saturated linear C₁-C₄ alkyl radical.

8. The regime or regimen as defined by claim 1, wherein formula (I):
Alk₁ and Alk₂ are each, independently of one another, a linear C₁-C₄ or branched C₃-C₄, saturated alkylene radical;
Ar₁ and Ar₂ are each a phenyl group;
R is a hydrogen atom or a linear C₁-C₄ or branched C₃-C₄, saturated alkyl radical, optionally substituted with a group selected from among —Ar₁, —OR₁, and —NR₁R₂;
R₁ and R₂ are each, independently of one another, a saturated linear C₁-C₄ alkyl radical.

9. The regime or regimen as defined by claim 1, wherein formula (I):
Alk₁ and Alk₂ are each, independently of one another, a linear C₁-C₄ or branched C₃-C₄, saturated alkylene radical;
Ar₁ and Ar₂ are each a phenyl group;
R is a hydrogen atom or a linear C₁-C₄ or branched C₃-C₄, saturated alkyl radical, optionally substituted with a group selected from among a phenyl radical, —NR₁R₂, and a hydrogen atom.

10. The regime or regimen as defined by claim 1, said at least one compound of formula (I) being selected from the group consisting of:
1-(2-phenylethyl)-4-[N-(3-phenylpropyl)]aminopiperidine;
1-(2-phenylethyl)-4-(N-benzyl)aminopiperidine;
1-(2-phenylethyl)-4-[N-(3,5-bistrifluoromethyl)benzyl]aminopiperidine;
1-(2-phenylethyl)-4-[N-(2-phenylethyl)]aminopiperidine;
1-(2-phenylethyl)-4-[N-(1 -phenylethyl)]aminopiperidine;
1-benzyl-4-[N-(1 -phenylethyl)]aminopiperidine;
1-[2-(3,4-dimethoxy)phenylethyl]-4-[N-(3-phenylpropyl)]aminopiperidine;
1-(3-phenylpropyl)-4-[N-(3-phenylpropyl)]aminopiperidine;
1-(2-phenylethyl)-4-[N-(4-phenylbutyl)]aminopiperidine;
1-(2-phenylethyl)-4-[N-(1-phenylpropyl)]aminopiperidine;
1-[2-(4-tert-butyl)phenylethyl]-4-[N-(3-phenylpropyl)]aminopiperidine;
1-(2-phenylethyl)-4-[N-(1S)-1-phenylethyl]aminopiperidine;
1-[2-(4-tert-butyl)phenylethyl]-4-[N-(1-phenylethyl)]aminopiperidine;
1-(4-phenylbutyl)-4-[N-(3-phenylpropyl)]aminopiperidine;
1-(3-phenylpropyl)-4-[N-(1-phenylpropyl)]aminopiperidine;
1-(3-phenylpropyl)-4-[N-(1 S)-1-phenylethyl]aminopiperidine;
1-(2-phenylethyl)-4-[N-methyl-N-(3-phenylpropyl)]aminopiperidine;
1-(2-phenylethyl)-4-[N,N-di(2-phenylethyl)]aminopiperidine;
1-(2-phenylethyl)-4-[N-ethyl-N-(1-phenylethyl)]aminopiperidine;
1-(2-phenylethyl)-4-[N-(2-phenylpropyl)-N-(1-phenylethyl)]aminopiperidine;
1-(2-phenylethyl)-4-[N-(3-phenylpropyl)-N-phenylethyl]aminopiperidine;
1-(2-phenylethyl)-4-[N-benzyl-N-(3-phenylpropyl)]aminopiperidine;
1-(2-phenylethyl)-4-[N-ethyl-N-(3-phenylpropyl)]aminopiperidine;
1-(2-phenylethyl)-4-[N-propyl-N-(3-phenylpropyl)]aminopiperidine;
1-(2-phenylethyl)-4-[N-butyl-N-3-(3-phenylpropyl)]aminopiperidine;
1-(2-phenylethyl)-4-[N-2-(3,4-dimethoxy)phenylethyl-N-(3-phenylpropyl)]aminopiperidine;
1-(2-phenylethyl)-4-[N-2-dimethylaminoethyl-N-(3-phenylpropyl)]aminopiperidine; and
1-(2-phenylethyl)-4-[N-2-methoxyethyl-N-(3-phenylpropyl)]aminopiperidine.

11. The regime or regimen as defined by claim 10, said at least one compound of formula (I) being selected from the group consisting of:
1-(2-phenylethyl)-4-[N-(3-phenylpropyl)]aminopiperidine,
1-(2-phenylethyl)-4-[N-(1-phenylethyl)]aminopiperidine,
1-benzyl-4-[N-(1-phenylethyl)]aminopiperidine,
1-(2-phenylethyl)-4-[N-(3-phenylpropyl)-N-phenylethyl]aminopiperidine,
1-(2-phenylethyl)-4-[N-butyl-N-3-(3-phenylpropyl)]aminopiperidine, and
1-(2-phenylethyl)-4-[N-2-dimethylaminoethyl-N-(3-phenylpropyl)]aminopiperidine.

12. The regime or regimen as defined by claim 11, said at least one compound of formula (I) being selected from the group consisting of:
1-(2-phenylethyl)-4-[N-(3-phenylpropyl)]aminopiperidine, and
1-(2-phenylethyl)-4-[N-(1-phenylethyl)]aminopiperidine.

13. The care and/or treatment composition as defined by claim 2, said at least one 4-aminopiperidine compound of formula (I) comprising from 10⁻³% to 10%, relative to the total weight of the composition.

14. The care and/or treatment composition as defined by claim 2, formulated as a hair cream or lotion, a shampoo, a conditioner, or a mascara for the hair or for the eyelashes.

15. The care and/or treatment composition as defined by claim 2, formulated as an aqueous, alcoholic or aqueous-alcoholic solution or suspension.

16. The regime or regimen as defined by claim 1, comprising co-administering to said individual at least one other ingredient selected from the group consisting of solvents, aqueous-phase or oily-phase thickeners or gelling agents, soluble dyestuffs, fillers, pigments, antioxidants, preservatives, fragrances, electrolytes, neutralizing agents, film-forming polymers, UV blockers, cosmetic and pharmaceutical active agents other than the compounds of formula (I), and mixtures thereof.

17. The regime or regimen as defined by claim 1, comprising co-administering to said individual at least one additional active compound that promotes the regrowth and/or limits the loss of keratin fibers.

18. The regime or regimen as defined by claim 17, said at least one additional active compound being selected from the group consisting of aminexil, 6-0-[(9Z,1 2Z)octadeca-9,12-dienoyl]hexapyranose, lipoxygenase inhibitors, bradykinin inhibitors, prostaglandins and derivatives thereof, prostaglandin receptor agonists or antagonists, non-prostanoic prostaglandin analogues, vasodilators, anti-androgens, cyclosporins and analogues thereof, anti-microbial agents, anti-inflammatories, retinoids, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic or acylhexosaccharidic acids, substituted arylethylenes, N-acylamino acids, flavonoids, ascomycin derivatives and analogues, histamine antagonists, saponins, proteoglycanase inhibitors, oestrogen agonists and antagonists, pseudoterines, cytokines and growth factor promoters, inhibitors of IL-1 or of IL-6, IL-10 promoters, TNF inhibitors, vitamins, benzophenones, hydantoin, retinoic acid, anti-pruriginous agents, anti-parasitic agents, anti-fungal agents, calcium antagonists, hormones, triterpenes, anti-androgenic agents, steroidal or non-steroidal 5α-reductase inhibitors, potassium-channel agonists, FP receptor antagonists, 15-hydroxyprostaglandin dehydrogenase inhibitors, and mixtures thereof.

19. The regime or regimen as defined by claim 1, comprising co-administering to said individual at least one active agent selected from the group consisting of proteins, protein hydrolysates, amino acids, polyols, urea, allantoin, sugars and sugar derivatives, water-soluble vitamins, plant extracts, hydroxy acids, retinol, tocopherol, derivatives of retinol or of tocopherol, essential fatty acids, ceramides, essential oils, salicylic acid derivatives, 5-n-octanoyl salicylic acid, hydroxy acid esters, phospholipids, and mixtures thereof.
